(19) United States
(12) Patent Application Publication
(10) Pub. No.: US 2015/0224195 A1
(43) Pub. Date: Aug. 13, 2015

(54) TOPICAL ECTOPARASITICIDE COMPOSITION

(71) Applicant: NORBROOK LABORATORIES LIMITED, Newry (GB)

(72) Inventors: William Blakely, Newry (GB); Lillian Cromie, Newry (GB)

(21) Appl. No.: 14/644,871

(22) Filed: Mar. 11, 2015

Related U.S. Application Data
(62) Division of application No. 12/922,221, filed on Nov. 29, 2010.

(30) Foreign Application Priority Data
Mar. 12, 2008 (GB) 0804619.5

(51) Int. Cl. A61K 47/14; A61K 9/00; A61K 45/06; A61K 31/231

(52) U.S. Cl. CPC A61K 47/14; A61K 31/231; A61K 9/0017; A61K 45/06

(57) ABSTRACT

A topical ectoparasiticide composition comprising an Insect Growth Regulator and at least one C₆-C₁₂ medium chain triglyceride wherein the composition comprises at least 60% (w/v) of the triglyceride based on the total composition.

The present invention relates to an ectoparasiticide composition for topical application comprising an Insect Growth Regulator, and its use in a method of treatment for the reduction or inhibition of the maturation of ectoparasites. In particular, the topical composition may be used in a method of treatment for reduction or inhibition of the maturation of fleas and ticks from infested animals.

Insect growth regulators (IGRs) like methoprene, hydroprene, kinoprene, fenoxycarb, pyriproxifen, cyromazine, dimilin and novaluron are a class of insecticides that inhibit chitin synthesis or the development of parasites from immature stages, like eggs and larvae, into the adults.

Common ectoparasiticides which may be treated with insect growth regulators include fleas and ticks, for example the Siphonaptera order and Ctencephalides Felis and Ctencephalides Canis, human fleas like Pulex Irritans, rat fleas like Xenopsylla Cheopis and ticks like those of cattle (e.g. Boophilus Microplus) and of dog (Rhipicephali Sanguineus).

Topical ectoparasiticide compositions are known, and may be in the form of spot-on products. Typically, only a few millilitres of such spot-on products containing an ectoparasiticide are administered onto a localised area on an animal's back. 24 hours after application, the complete skin surface of the animal is protected by the ectoparsiticide. It is believed that upon application, the insecticide is adsorbed onto the skin surface and solubilised in the skin sebum from where it spreads along the surface by diffusion. Resevoirs of the insecticide are believed to form in the sebaceous glands thereby providing a supply of the drug over a long period of time, e.g. from 6 to 8 weeks of protection.

Examples of formulations containing methoprene which are effective against ticks include U.S. Pat. No. 5,194,264 which describes an aqueous/polar solvent methoprene composition. U.S. Pat. No. 6,492,419 discloses a composition with an Insect Growth Regulator (IGR) in a vehicle comprising a suspending agent, an anionic surfactant, a non-ionic surfactant or mixtures thereof, and an aqueous carrier.

A methoprene fipronil combination spot-on product exists (Frontline™ Plus) which solubilises both products in ethanol and a number of excipients including povidone, diethyleneglycolmonoethylether and antioxidants required for stability and to inhibit crytalisation of the actives, especially on the skin surface of the animal.

The known formulations typically require mixtures of solvents and/or the presence of one or more crystallisation inhibitors in order to provide stable compositions in which the active (IGR) is prevented from crystallising out on the skin surface of the treated animal.

It is an object of the present invention to provide a stable topical composition for application to humans or animals comprising an Insect Growth Regulator, especially methoprene, which preferably does not require the presence of adjuvants and/or crystallisation inhibitors in a solvent system, and still provides efficacious levels of insecticide activity over the surface of the human or animal treated for a number of weeks.

In a first aspect of the present invention there is provided a topical ectoparasiticide composition comprising an Insect Growth Regulator and at least one C₆-C₁₂ medium chain triglyceride, wherein the composition comprises at least 60% (w/v) of the triglyceride based on the total composition.

In a second aspect of the present invention there is provided a composition as described herein for use in a method of treatment of the human or animal body by therapy.

In a third aspect of the present invention there is provided a composition as described herein for use in a method of treatment for the reduction or inhibition of juvenile ectoparasite maturation from the skin of an animal, wherein the composition is applied topically to the skin of the animal.

In a fourth aspect of the present invention there is provided the use of a composition as described herein for the reduction or inhibition of juvenile ectoparasite maturation from the skin of an animal or from the environment of an animal.

In a fifth aspect of the present invention there is provided a kit comprising separately, in the same packaging, at least one container containing the composition as described herein and at least one container containing at least one adjuvant selected from anti-oxidants and other actives.

The present inventors have surprisingly found that by using a solvent comprising at least 60% (w/v) of the least one C₆-C₁₂ medium chain triglyceride(s) stable topical compositions may be produced without the need to include additional adjuvants or further crystallisation inhibitors. The formation of stable topical compositions without crystallisation inhibitors is advantageous because the product is easier, faster and cheaper to make, whilst still providing an efficient and effective topical composition for the reduction or elimination of ectoparasites. It has surprisingly been found that such compositions, even without the presence of additional crystallisation inhibitors, do not crystallise on the skin of an animal after application. These compositions have also been found to have good storageability. Furthermore these compositions do not cause, or cause reduced skin irritation at the site of application.

Each aspect as defined herein may be combined with any other aspect or aspects unless clearly indicated to the contrary. In particular any feature indicated as being preferred or advantageous may be combined with any other feature or features indicated as being preferred or advantageous.

Preferably the Insect Growth Regulator is selected from methoprene, hydroprene, kinoprene, fenoxycarb, pyriproxifen, cyromazine, dimilin, novaluron and mixtures of two or more thereof. Most preferably the Insect Growth Regulator is methoprene.

The Insect Growth Regulator may be present from 0.1% to 100% (weight/volume) w/v, preferably it is present between from 1 to 40% w/v, more preferably from 5 to 20% most preferably from 8 to 15% w/v, even more preferably it is present at 12% w/v.

As used herein the term “C₆-C₁₂ medium chain triglyceride” includes all pharmaceutically or veterinary acceptable saturated or unsaturated aliphatic triglycerides having from 6 to 12 carbon atoms in their chain.

The C₆-C₁₂ medium chain triglyceride may be a single triglyceride or a mixture of two or more thereof. Examples are C₆, C₈, C₁₀ and/or C₁₂ chain triglycerides. Suitable triglycerides are neobee oil, coconut oil and palm kernel oil.

Preferably the medium-chain triglyceride is derived from cotton seed oil.

Preferably the composition comprises at least 80% (w/v), more preferably at least 90% (w/v), of the least one medium chain triglyceride. The composition may comprise at least 80% (w/v), more preferably at least 90% (w/v), of a specific medium chain triglyceride, for example, a C₆, C₈, C₁₀ or C₁₂ chain triglyceride based on the total composition. The composition may comprise at least 80% (w/v), more preferably at least 90% (w/v) of at least two or more medium chain triglycerides based on the total composition.

Preferably the composition of the present invention is a non-aqueous composition. Preferably the composition comprises less than 1% (w/v), more preferably less than 0.5% (w/v) water based on the total composition. Most preferably the composition does not comprise any water.

Other suitable solvents may be present in the topical composition. Suitable other solvents include, but are not limited to acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone, in particular N-methylpyrrolidone, diethylene glycol monoethyl ether, ethylene glycol, diethyl phthalate, and mixtures of two or more thereof. The preferred additional solvents are ethanol, isopropanol, benzyl alcohol, or butanol.

Preferably the composition of the present invention is free of crystallisation inhibitors. This has the advantage that the composition may be made more cheaply and efficiently, whilst still being effective.

Advantageously, the composition of the present invention comprises less than 25% (w/v) of crystallisation inhibitor, more preferably less than 10% (w/v), more preferably still less than 1% (w/v) based on the total composition.

As used herein the term “crystallisation inhibitor” may be used to mean an agent or substance which inhibits crystal formation of the insect growth inhibitor in the composition. The crystallisation inhibitor preferably corresponds to a test in which 10 ml of the composition comprising 10% (w/v) of inhibitor is placed in a glass slide at 20° C. for 24 hours. The slide is then observed with the naked eye. Acceptable inhibitors are those whose addition provides few or no crystals, and in particular less than 10 crystals, preferably less than 5 crystals, more preferably 0 crystals. As used herein the term “crystallisation inhibitor” does not include fatty acids, or C₄-C₂₄ aliphatic acids.

In an alternative embodiment, the composition of the present invention may comprise at least one crystallisation inhibitor. Suitable crystallisation inhibitors are known in the art and include, but are not limited to polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, sodium carboxymethylcellulose; acrylic derivatives such as methacrylates and the like, alkyl sulphates, in particular sodium lauryl sulphate and sodium cetyl sulphate; sodium dodecylbenzenesulphonate, sodium dioctylsulphosuccinate; cationic surfactants such as water-soluble quaternary ammonium salts of formula N⁺R′R″R″′R″″Y⁻ in which the radicals R are hydrocarbon radicals, optionally hydroxylated, and Y⁻ is an anion of a strong acid such as halide, sulphate and sulphonate anions; cetyltrimethylammonium bromide is among the cationic surfactants which can be used, amine salts of formula NR′R″R′″ in which the radicals R are optionally hydroxylated hydrocarbon radicals; octadecylamine hydrochloride is among the cationic surfactants which can be used, nonionic surfactants such as optionally polyoxyethylenated sorbitan esters, in particular polysorbate 80, polyoxyethylenated alkyl ethers; polyethylene glycol stearate, polyoxyethylenated derivatives of castor oil, polyglycerol esters, polyoxyethylenated fatty alcohols, copolymers of ethylene oxide and propylene oxide, amphoteric surfactants such as lauryl-substituted betaine compounds, or preferably a mixture of at least two of these crystallization inhibitors. Preferably the crystallisation inhibitor is polyvinylpyrrolidone, polyvinyl alcohols, polyethylene glycol, benzyl alcohol and/or lecithin.

The composition may comprise at least one adjuvant selected from anti-oxidants and other actives.

Suitable antioxidants include, but are not limited to butylated hydroxyanisole (BHA), butylated hydroxytoluene, ascorbic acid, alpha, beta or gamma tocopherol, sodium metabisulphite, propyl gallate, sodium thiosulphate, and mixtures of two or more thereof. Preferred antioxidants are butylated hydroxyanisole (BHA) and butylated hydroxytoluene. Addition of antioxidants may be advantageous in extending the shelf-life of the compositions.

Preferably in the composition anti-oxidants are present in a concentration of from 0.005 to 1% (w/v) based on the total composition, more preferably from 0.01 to 0.05% (w/v).

The other-actives may be selected from one or more of other phenyl pyrazoles, spinosads, non-steroidal anti-inflammatory drugs (NSAIDs), steroidal anti-inflammatory drugs, macrocyclic lactones, milbemycine oximes, insect growth regulators, chitin synthesis inhibitors and RNA inhibitors.

Suitable non-steriodal anti-inflammatory drugs (NSAID) include, but are not limited to, ibuprofen, carprofen, meloxicam and acetaminophen.

Suitable steroidal anti-inflammatory drugs include, but are not limited to, codeine, cortisone and hydro-cortisone.

Examples of Milbemycine oximes include, but are not limited to, avermectins, ivermectin, selamectin, moxidectin, abamectin and doramectin.

Suitable insect growth regulators include, but are not limited to, methoprene, pyriproxyfen, kinoprene and fenoxycarb.

Examples of chitin synthesis inhibitors include, but are not limited to, triflumuron, lufenuron, chlorofluazuron and fluazuron.

Suitable amounts of the other-actives will depend on the active in question. Typically the other-actives may be present in a concentration of from 0.1 to 30% (w/v) based on the total composition, preferably from 5 to 20% (w/v).

Other actives include agents which, with the composition of the present invention may be sprayed, squirted, or rubbed on to the skin. These include, for example, conventional propellent gases required for spray cans, such as propane, butane, dimethyl ether, CO₂, or halogenated lower alkyl gases (for example, halogenated C₁-C₄ alkyls), and mixtures of two or more thereof.

In one embodiment of the present invention the composition consists of an Insect Growth Regulator and a solvent comprising at least one C₆-C₁₂ medium chain triglyceride.

The compositions according to the invention are usually prepared by simply mixing the constituents as defined above. Advantageously, to begin with, the insect growth regulator is mixed into the main solvent, and the other ingredients or adjuvants are subsequently added.

The compositions according to the invention are typically intended for pets, in particular cats and dogs, and are generally applied by deposition on the skin (“spot on” or “pour on” application). This is generally a localized application to a region with a surface area of less than 10 cm², typically between 5 and 10 cm². The composition may, for example, by applied at one, two or more points and is preferably localized between the animal's shoulders. After deposition, the composition diffuses, in particular over the animal's entire body, and then dries, without crystallizing or changing the appearance (in particular there is an absence of any whitish deposit or of any dusty appearance) or the feel of the coat. The composition of the present invention may be a spot-on or a spray-on formulation.

The composition of the present invention may be in the form of a concentrated emulsion, microemulsion, suspension, or solution for spot-on application to an animal. In less preferred embodiments the composition may be in the form of a spray, an emulsion, microemulsion, suspension, or solution of the pour-on-type, an oil, a cream, an ointment, or any other fluid formulation for topical administration.

The compositions according to the present invention are particularly advantageous on the grounds of their efficacy, their speed of action and the pleasant appearance of the animal's hair after application and drying.

It is preferable that the composition of the present invention is administered every 4 weeks or even more preferably every 8 or 12 weeks on small animals, such as cats and dogs.

The volume applied to a dog is typically from 0.25 to 3 ml and to a cat is typically from 0.25 to 1 ml.

The composition of the present invention may be used to treat insect infestation on humans, large and small animals, birds and reptiles. Preferably the animal to be treated is a human, cow, horse, bird or small animal. Most preferably it is a cat or a dog. The larger the animal to be treated, the larger the dose volume of the composition to be applied. The composition of the present invention is especially suitable for administration to dogs and cats.

The composition of the present invention is preferably administered in order to provide doses of from 1 to 30 mg/kg of the insect growth regulator per kg of animal body weight, more preferably from 5 to 25 mg/kg, more preferably still from 10 to 20 mg/kg.

The composition of the present invention may be used to improve the appearance and texture of an animal's coat by elimination or reduction of mature entoparasites therefrom and any consequential irritation caused, however slight, to the infected animal. One object of the present invention is to provide a non-therapeutic method of cleaning animal hairs and skin by the reduction or elimination of mature parasites which are present in the animal hair or skin. The treated animals have hair that has a more pleasant look and feel.

Additionally, the compositions of the current invention may be used prophylactically in order to inhibit or reduce maturation of juvenile ectoparasites like fleas or even ticks. The compositions may be used such that the treated animal are used as vectors in order to eradicate or reduce insects (for example ticks) from the animals environment, e.g. like bedding, carpet, floors and walls.

In one embodiment, the present invention provides a therapeutic treatment, and the composition may be used in a method of treatment for the inhibition of juvenile ectoparasite maturation from the skin of an animal, wherein the composition is applied topically to the skin of the animal. The process described herein may be used to control ectoparasites, and in particular ticks.

In one aspect of the present invention there is provided the use of a composition as described herein in the manufacture of a medicament for the inhibition or reduction of juvenile ectoparasite maturation from the skin of an animal.

In further embodiment the present invention provides a method for the inhibition or reduction of juvenile ectoparasite maturation from the skin of an animal, the method comprising applying the topical composition as defined herein to the skin of an animal. Preferably the topical composition is in the form of a spot-on composition. Preferably the composition is applied between the shoulders of the animal. Preferably the animal is a dog or a cat. Preferably, the composition comprises methoprene. Preferably the composition is applied in unit dosage form.

In one aspect of the present invention there is provided a kit comprising separately, in the same packaging, at least one container containing the composition as defined herein and at least one container containing at least one adjuvant selected from anti-oxidants and other actives. The other active is selected from one or more phenyl pyrazoles, spinosads, non-steroidal anti-inflammatory drugs (NSAIDs), steroidal anti-inflammatory drugs, macrocyclic lactones, milbemycine oximes, other insect growth regulators, chitin synthesis inhibitors and RNA inhibitors. Preferably the other active is an insect growth regulator.

Optionally a further active agent may be applied to the skin of an animal at the same time as, before, or after application of the topical composition of the present invention. The further active agent may be applied at the same, or different location on an animal as the composition of the present invention. Preferably, the further active is an insect growth regulator. The other active may be selected from one or more phenyl pyrazoles, spinosads, non-steroidal anti-inflammatory drugs (NSAIDs), steroidal anti-inflammatory drugs, macrocyclic lactones, milbemycine oximes, other insect growth regulators, chitin synthesis inhibitors and RNA inhibitors. The further active may be applied concomitantly or alternately. For example the actives may be applied using a dual applicator in which the compositions containing the two actives are contained separately, but allows the controlled release of one or both of the actives concomitantly or alternately.

The present invention will be further illustrated with reference to the following non-limiting Examples.

Compositions according to the present invention were made containing the following concentrations (W/V):

### EXAMPLE 1

Methoprene 12% (w/v)

Neobee oil q.s. (quantity sufficient) 100%

### Skin Tolerance Test:

Skin tolerance tests were carried out on cats and dogs.

No Irritation Observed

No Crystallisation Observed

1-17. (canceled)

18. A method of reducing or inhibiting juvenile ectoparasite maturation from the skin of an animal, the method comprising topically administering an effective amount of a composition comprising
an Insect Growth Regulator selected from the group consisting of methoprene, hydroprene, and kinoprene, and
at least one C6-C12 medium chain triglyceride wherein the composition comprises at least 60% (w/v) of the triglyceride based on the total composition,
to an animal in need thereof.

19. The method of claim 18 wherein the composition comprises at least 80% (w/v) of the triglyceride based on the total composition.

20. The method of claim 18 wherein the composition comprises at least 90% (w/v) of the triglyceride based on the total composition.

21. The method of claim 18 wherein the composition consists of an Insect Growth Regulator selected from the group consisting of methoprene, hydroprene, and kinoprene and the triglyceride.

22. The method of claim 18 wherein the triglyceride comprises a caproic, caprylic, capric or lauric acid triglyceride or a mixture of two or more thereof.

23. The method of claim 18 wherein the Insect Growth Regulator is methoprene .

24. The method of claim 18 wherein the composition further comprises at least one adjuvant selected from anti-oxidants and other actives.

25. The method of claim 24 wherein the other active is selected from one or more of phenyl pyrazoles, spinosads, non-steroidal anti-inflammatory drugs (NSAIDs), steroidal anti-inflammatory drugs, macrocyclic lactones, milbemycine oximes, other insect growth regulators, chitin synthesis inhibitors and RNA inhibitors.

26. The method of claim 18 wherein the Insect Growth Regulator is present in a concentration of from 0.1 to 40% (w/v) based on the total composition.

27. The method of claim 18 wherein the composition is applied topically in the form of a spot-on or spray-on formulation for an animal.
